(19) United States
(12) Patent Application Publication
(10) Pub. No.: US 2026/0183546 A1
(43) Pub. Date: Jul. 2, 2026

(54) PHYSIOLOGICAL SIGNAL DETECTION AND ELECTRICAL STIMULATION SYSTEM, ELECTRICAL STIMULATION DEVICE THEREOF AND METHOD THEREOF

(71) Applicant: INDUSTRIAL TECHNOLOGY RESEARCH INSTITUTE, Hsinchu (TW)

(72) Inventors: Ming-Ya HUNG, Taoyuan City (TW); Chen-Liang LIN, Chiayi City (TW); Yu-Yuan CHEN, Zhubei City (TW); Po-Yen LIU, Taoyuan City (TW); Chun-Yu CHAN, Hsinchu City (TW); Jun-Chao ZHAO, Tainan City (TW)

(73) Assignee: INDUSTRIAL TECHNOLOGY RESEARCH INSTITUTE, Hsinchu (TW)

(21) Appl. No.: 19/005,532

(22) Filed: Dec. 30, 2024

(51) Int. Cl. A61N 1/36

(52) U.S. Cl. CPC A61N 1/36031

(57) ABSTRACT

An electrical stimulation device includes N electrodes, N first electrode switches, N second electrode switches, a first control module and a second control module. N is a positive integer equal to or greater than 2. The N first electrode switches are respectively coupled to the N electrodes. The N second electrode switches are respectively coupled to the N electrodes. The first control module is coupled to the N first electrode switches. The second control module is coupled to the N second electrode switches. The N first electrode switches and the first control module are coupled to a common contact point, and the N second electrode switches and the second control module are coupled to a common contact point.

### TECHNICAL FIELD

The technical field relates to a physiological signal detection and an electrical stimulation system, an electrical stimulation device thereof and a method thereof.

### BACKGROUND

At present, in the medical treatment for human diseases or discomforts, electromyography devices are first used to measure the myoelectric signal of the human body. Different myoelectric signals respond to different physical conditions. Then, the doctor decides the treatment plan according to the electromyographic signal. However, such method requires the replacement of different equipment and is difficult to provide a comprehensive solution for home exercise training required by a clinical therapist and a patient.

### SUMMARY

According to an embodiment, a physiological signal detection and electrical stimulation system is provided. The physiological signal detection and electrical stimulation system includes a physiological signal processing unit, an electrical stimulation device and a processing device. The physiological signal processing unit is configured to generate a physiological signal. The electrical stimulation device includes N electrodes, a first circuit assembly and a second circuit assembly. N is a positive integer equal to or greater than 2. The first circuit assembly includes N first electrode switches and a first control module, the first electrode switches are coupled to the N electrodes respectively, and the first control module is coupled to the N first electrode switches. The second circuit assembly includes N second electrode switches and a second control module, N second electrode switches are coupled to the N electrodes respectively, and the second control module is coupled to the N second electrode switches. The processing device is electrically connected to the electrical stimulation device and the physiological signal processing unit, and configured to: control the N first electrode switches, the N second electrode switches, the first control module and the second control module to generate a current flowing along an electrical stimulation path according to the physiological signal. The N first electrode switches and the first control module share a contact point, and the N second electrode switches and the second control module share another contact point.

According to another embodiment, an electrical stimulation device is provided. The electrical stimulation device includes N electrodes, a first circuit assembly and a second circuit assembly. N is a positive integer equal to or greater than 2. The first circuit assembly includes N first electrode switches and a first control module, the N first electrode switches are coupled to the N electrodes respectively, and the first control module is coupled to the N first electrode switches. The second circuit assembly includes N second electrode switches and a second control module, the second electrode switches are coupled to the N electrodes respectively, and the second control module is coupled to the N second electrode switches. The N first electrode switches and the first control module share a contact point, and the N second electrode switches and the second control module share another contact point.

According to another embodiment, a physiological signal detection and electrical stimulation method is provided. The physiological signal detection and electrical stimulation method includes the following steps: generating a physiological signal by a physiological signal processing unit of a physiological signal detection and electrical stimulation system, wherein the physiological signal detection and electrical stimulation system further comprises an electrical stimulation device and a processing device, the electrical stimulation device comprises N electrodes, a first circuit assembly and a second circuit assembly, the first circuit assembly comprises N first electrode switches and a first control module, the second circuit assembly comprises N second electrode switches and a second control module, wherein N is a positive integer equal to or greater than 2, the N first electrode switches are coupled to the N electrodes respectively, the N second electrode switches are coupled to the N electrodes respectively, the first control module is coupled to the N first electrode switches, the second control module is coupled to the N second electrode switches, the N first electrode switches and the first control module share a contact point, the N second electrode switches and the second control module share another contact point, and the processing device is electrically connected to the electrical stimulation device and the physiological signal processing unit; and controlling the N first electrode switches, the N second electrode switches, the first control module and the second control module to generate a current flowing along an electrical stimulation path according to the physiological signal.

According to another embodiment, an electrical stimulation device is provided. The electrical stimulation device includes M circuit assemblies. M is a positive integer equal to or greater than 2, and each of the M circuit assemblies includes P electrodes, P electrode switches and a control module. P is a positive integer equal to or greater than 2. The P electrode switches are coupled to the P electrodes respectively. The control module is coupled to the P electrode switches. A current is allowed to be transmitted from at least one of the P electrodes of one of the M circuit assemblies to at least one of the P electrodes of at least one of the others of the M circuit assemblies.

### BRIEF DESCRIPTION OF THE DRAWINGS

FIG. 1A illustrates a schematic diagram of a physiological signal detection and electrical stimulation product according to an embodiment of the present disclosure;

FIG. 1B illustrates a functional block diagram of a physiological signal detection and electrical stimulation system of the physiological signal detection and electrical stimulation product in FIG. 1A;

FIGS. 2A and 2B illustrates a circuit schematic diagram of an electrical stimulation device in FIG. 1B;

FIG. 3A illustrates a schematic diagram of a first electrical stimulation mode according to an embodiment of the present disclosure;

FIG. 3B illustrates a schematic diagram of a second electrical stimulation mode according to an embodiment of the present disclosure;

FIG. 4A illustrates a schematic diagram of a third electrical stimulation mode according to an embodiment of the present disclosure;

FIG. 4B illustrates a schematic diagram of a fourth electrical stimulation mode according to an embodiment of the present disclosure;

FIG. 5A illustrates a schematic diagram of a fifth electrical stimulation mode according to an embodiment of the present disclosure;

FIG. 5B illustrates a schematic diagram of a sixth electrical stimulation mode according to an embodiment of the present disclosure;

FIG. 6A illustrates a schematic diagram of a seventh electrical stimulation mode according to an embodiment of the present disclosure;

FIG. 6B illustrates a schematic diagram of an eighth electrical stimulation mode according to an embodiment of the present disclosure;

FIG. 7 illustrates a circuit schematic diagram of an electrical stimulation device according to another embodiment of the present disclosure; and

FIG. 8 illustrates a circuit schematic diagram of an electrical stimulation device according to another embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically illustrated in order to simplify the drawing.

### DETAILED DESCRIPTION

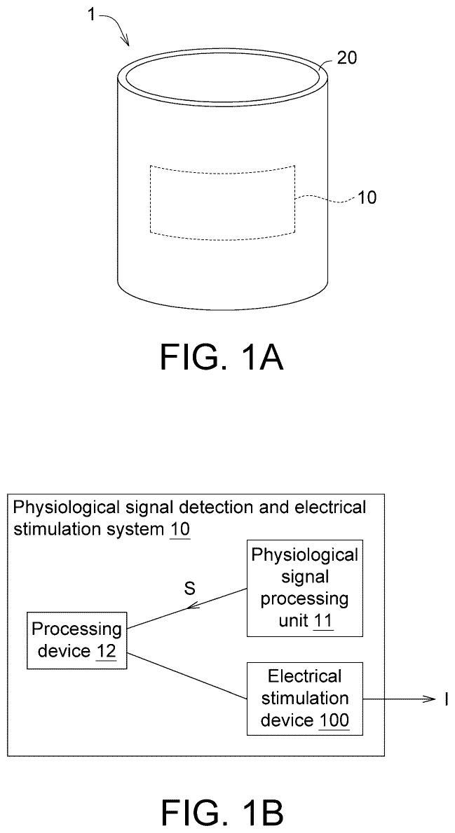

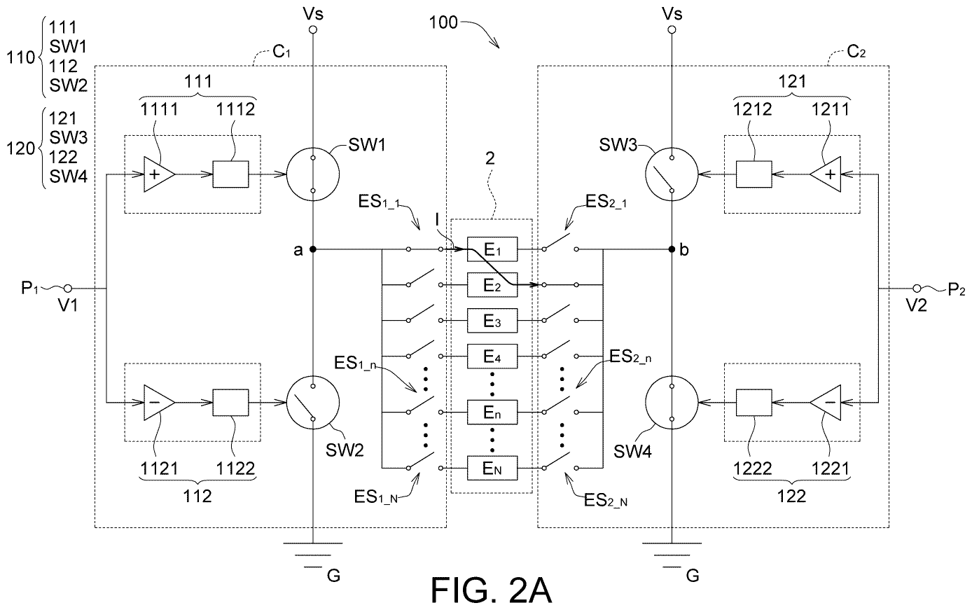

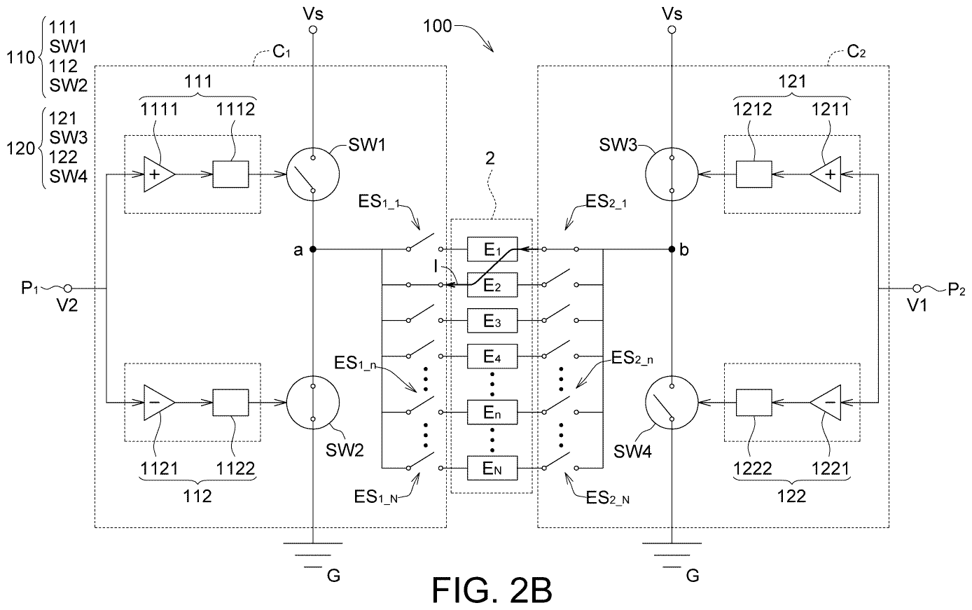

Referring to FIGS. 1A, 1B, 2A and 2B, FIG. 1A illustrates a schematic diagram of a physiological signal detection and electrical stimulation product 1 according to an embodiment of the present disclosure, FIG. 1B illustrates a functional block diagram of a physiological signal detection and electrical stimulation system 10 of the physiological signal detection and electrical stimulation product 1 in FIG. 1A, FIGS. 2A and 2B illustrates a circuit schematic diagram of an electrical stimulation device 100 in FIG. 1B.

As illustrated in FIGS. 1A and 1B, the physiological signal detection and electrical stimulation product 1 is, for example, a wearable product, which may be disposed on a living body (or an organism) 2 (the living body 2 is illustrated in FIG. 2A) to detect the physiological signal S of the living body 2 and apply a current I to the living body 2 for electrically stimulating the living body 2. The living body 2 is, for example, a human body. The physiological signal detection and electrical stimulation product 1 includes a physiological signal detection and electrical stimulation system 10 and a carrier 20. The carrier 20 is made of, for example, fibers (e.g., elastic fibers), cloth, compounds, or other materials (e.g., woven) suitable for contacting the living body 2. Furthermore, the carrier 20 is, for example, kneepad, sock, clothing, pant, neckband, glove, etc. The physiological signal detection and electrical stimulation system 10 may be disposed inside or outside the carrier 20 and may contact the living body 2 to detect the physiological signal S of the living body 2. In an embodiment, the physiological signal detection and electrical stimulation system 10 may be worn on the human body's knee, ankle, wrist, neck, waist, or other part that require electrical stimulation.

As illustrated in FIGS. 1A and 1B, the physiological signal detection and electrical stimulation system 10 includes a physiological signal processing unit 11, a processing device 12 and an electrical stimulation device 100. In an embodiment, the processing device 12 may be disposed outside the physiological signal detection and electrical stimulation system 10 or outside the physiological signal detection and electrical stimulation product 1, and communicate with the physiological signal processing unit 11 and the electrical stimulation device 100 through a wired communication technology or a wireless communication technology. The physiological signal processing unit 11 may detect the physiological information of the living body 2 (the living body 2 is illustrated in FIG. 2A), and accordingly generate the physiological signal S, such as a myoelectric signal. The processing device 12 is electrically connected to the electrical stimulation device 100 and the physiological signal processing unit 11 and may control the electrical stimulation device 100 to generate the current I according to the physiological signal S for electrically stimulating the living body 2. In addition, the processing device 12 and the physiological signal processing unit 11 which include at least one physical circuit are, for example, semiconductor wafers or semiconductor packages formed by a semiconductor process.

As illustrated in FIGS. 2A and 2B, the electrical stimulation device 100 includes, for example, an electrical stimulation circuit. The electrical stimulation circuit includes N electrodes E₁ to E_{N}, a first circuit assembly C1 and a second circuit assembly C2. The first circuit assembly C1 includes N first electrode switches ES_{1_1} to ES_{1_N} and a first control module 110. The second circuit assembly C2 includes N second electrode switches ES_{2_1} to ES_{2_N} and a second control module 120. N is a positive integer equal to or greater than 2. N first electrode switches ES_{1_1} to ES_{1_N} are coupled to N electrodes E₁ to E_{N} respectively. N second electrode switches ES_{2_1} to ES_{2_N} are coupled to N electrodes E₁ to E_{N} respectively. The N first electrode switches ES_{1_1} to ES_{1_N} share a contact point a with the first control module 110, and the N second electrode switches ES_{2_1} to ES_{2_N} share a contact point b with the second control module 120. The processing device 12 is configured to control the action modes of the N first electrode switches ES_{1_1} to ES_{1_N}, the N second electrode switches ES_{2_1} to ES_{2_N}, the first control module 110 and the second control module 120 according to the physiological signal S, so as to generate the current I flowing along an electrical stimulation path. As a result, by controlling the action modes of the N first electrode switches ES_{1_1} to ES_{1_N}, the N second electrode switches ES_{2_1} to ES_{2_N}, the first control module 110 and the second control module 120, the path of the current I may be controlled to obtain the desired electrical stimulation effect. In addition, in the present embodiment, the detection of physiological signals and the application of electrical stimulation may be integrated into the same device (for example, the physiological signal detection and electrical stimulation system 10), thereby increasing the convenience of use.

As illustrated in FIGS. 2A and 2B, the N electrodes E₁ to E_{N} may be adjacent to or in contact with the living body 2, so that the current I may stimulate the living body 2 through the electrodes.

In an embodiment, the first circuit assembly C1 is, for example, a first H half-bridge circuit, and the second circuit assembly C2 is, for example, a second H half-bridge circuit, wherein the first H half-bridge circuit and the second H half-bridge circuit constitute an H-bridge circuit set. Although the electrical stimulation device 100 in the embodiment of the present disclosure uses an H-bridge circuit set as an example, in other embodiments, the electrical stimulation device 100 may include M H half-bridge circuits, wherein M is, for example, a positive integer equal to or greater than 2, and at least one of the electrodes E₁ to E_{N} of one of the M H half-bridge circuits may be connected in series or in parallel with at least one of the electrodes E₁ to E_{N} of another of the M H half-bridge circuits.

As illustrated in FIGS. 2A and 2B, the first circuit assembly C1 and the second circuit assembly C2 share N electrodes E₁ to E_{N}. The half-bridge circuit set at least includes N electrodes E₁ to E_{N} and 2N electrode switches (i.e., N first electrode switches ES_{1_1} to ES_{1_N} and N second electrode switches ES_{2_1} to ES_{2_N}). In addition, the aforementioned physiological signal processing unit 11 and the electrical stimulation device 100 may also share at least one of the N electrodes E₁ to E_{N}, or the detection electrodes of the physiological signal processing unit 11 and the electrodes E₁ to E_{N} of the electrical stimulation device 100 may be separately disposed (i.e. do not share the electrode).

As illustrated in FIGS. 2A and 2B, the first electrode switches ES_{1_1} to ES_{1_N} are coupled to the N electrodes E₁ to E_{N} respectively, and the second electrode switches ES_{2_1} to ES_{2_N} are coupled to the N electrodes E₁ to E_{N} respectively. For example, the electrode E₁ is coupled to the first electrode switch ES_{1_1} and the second electrode ES_{2_1}, the electrode E₂ is coupled to the first electrode switch ES_{1_2} and the second electrode switch ES_{2_2}, . . . , and so on, the electrode E_{N} is coupled to the first electrode switch ES_{1_N} and the second electrode switch ES_{2_N}. In an embodiment, the two position points of the electrode Eₙ are respectively coupled to the first electrode switch ES_{1_n} and the second electrode switch ES_{2_n}, and the two position points are respectively located on two opposite sides, two adjacent sides, or the same side of the electrode Eₙ.

As illustrated in FIGS. 2A and 2B, the first control module 110 includes a first switch SW1, a second switch SW2, a first driving circuit 111 and a second driving circuit 112. The first driving circuit 111 is electrically connected to the first switch SW1, and the second driving circuit 112 is electrically connected to the second switch SW2. The first driving circuit 111 may control the first switch SW1 to be turned on or off, and the second driving circuit 112 may control the second switch SW2 to be turned on or off for controlling the current path. The second control module 120 includes a third switch SW3, a fourth switch SW4, a third driving circuit 121 and a fourth driving circuit 122. The third driving circuit 121 is electrically connected to the third switch SW3, and the fourth driving circuit 122 is electrically connected to the fourth switch SW4. The third driving circuit 121 may control the third switch SW3 to be turned on or off, and the fourth driving circuit 122 may control the fourth switch SW4 to be turned on or off for controlling the current path.

As illustrated in FIGS. 2A and 2B, the second switch SW2 and the fourth switch SW4 may be electrically connected to a ground potential G. The voltage source Vs may be supplied to the first control module 110 and the second control module 120. For example, the voltage source Vs is electrically connected to the first switch SW1 of the first control module 110 and the third switch SW3 of the second control module 120. When the first switch SW1 is turned on, the voltage source Vs may apply a voltage to the corresponding electrode through the first switch SW1 and at least one conductive one of the first electrode switches ES_{1_1} to ES_{1_N} that is turned on. When the second switch SW2 is turned on, the ground potential G may be applied to the corresponding electrode through the second switch SW2 and at least one conductive one of the first electrode switches ES_{1_1} to ES_{1_N} that is turned on. When the third switch SW3 is turned on, the voltage source Vs may apply the voltage to the corresponding electrode through the third switch SW3 and at least one conductive one of the second electrode switches ES_{2_1} to ES_{2_N} that is turned on. When the fourth switch SW4 is turned on, the ground potential G may be applied to the corresponding electrode through the fourth switch SW4 and at least one conductive one of the second electrode switches ES_{2_1} to ES_{2_N} that is turned on.

As illustrated in FIGS. 2A and 2B, a first input port P₁ is coupled to the first control module 110, for example, the first driving circuit 111 and the second driving circuit 112 of the first control module 110. By controlling the voltage of the first input port P₁, the first switch SW1 may be controlled to be on or off, and the second switch SW2 may be controlled to be on or off. The second input port P₂ is coupled to the second control module 120, for example, the third driving circuit 121 and the fourth driving circuit 122 of the second control module 120. By controlling the voltage of the second input port P₂, the third switch SW3 may be controlled to be turned on or off, and the fourth switch SW4 may be controlled to be turned on or off.

As illustrated in FIGS. 2A and 2B, the first driving circuit 111 includes a first non-inverter 1111 and a first driver 1112, wherein the first non-inverter 1111 is electrically connected to the first driver 1112. The first switch SW1 is, for example, a transistor, and the first driver 1112 is, for example, a gate driver. The second driving circuit 112 includes a first inverter 1121 and a second driver 1122, wherein the first inverter 1121 is electrically connected to the second driver 1122. The second switch SW2 is, for example, a transistor, and the second driver 1122 is, for example, a gate driver. The third driving circuit 121 includes a second non-inverter 1211 and a third driver 1212, wherein the second non-inverter 1211 is electrically connected to the third driver 1212. The third switch SW3 is, for example, a transistor, and the third driver 1212 is, for example, a gate driver. The fourth driving circuit 122 includes a second inverter 1221 and a fourth driver 1222, wherein the second inverter 1221 is electrically connected to the fourth driver 1222. The fourth switch SW4 is, for example, a transistor, and the fourth driver 1222 is, for example, a gate driver.

The processing device 12 is further configured to control one of the first switch SW1 and the second switch SW2 to turn on and the other of the first switch SW1 and the second switch SW2 to turn off by using one of the first level voltage V1 and the second level voltage V2; and control one of the third switch SW3 and the fourth switch SW4 to turn on and the other of the third switch SW3 and the fourth switch SW4 to turn off by using the other of the first level voltage V1 and the second level voltage V2. The first level voltage V1 is different from the second level voltage V2. In the present embodiment, the first level voltage V1 is higher than the second level voltage V2, that is, the first level voltage V1 has a high potential, and the second level voltage V2 has a low potential.

For example, as illustrated in FIG. 2A, the processing device 12 inputs the first input port P₁ with the first level voltage V1, and the first non-inverter 1111 outputs a control signal to the first driver 1112 according to the first level voltage V1, the first driver 1112 controls the first switch SW1 to turn on, and the first inverter 1121 outputs the control signal to the second driver 1122 according to the first level voltage V1, and the second driver 1122 controls the second switch SW2 to turn off. The processing device 12 inputs the second input port P₂ with the second level voltage V2. The second non-inverter 1211 outputs the control signal to the third driver 1212 according to the second level voltage V2, accordingly the third driver 1212 controls the third switch SW3 to turn off, and the second inverter 1221 outputs the control signal to the fourth driver 1222 according to the second level voltage V2, and the fourth driver 1222 controls the fourth switch SW4 to turn on accordingly. As a result, the voltage source Vs may form an electrical stimulation path (from high potential to low potential) through sequentially the first switch SW1, at least one conductive one of the first electrode switches ES_{1_1} to ES_{1_N} that is turned on, the corresponding electrode, at least one conductive one of the second electrode switches ES_{2_1} to ES_{2_N} that is turned on and the fourth switch SW4.

In the present embodiment, as illustrated in FIG. 2A, the voltage source Vs forms an electrical stimulation path through sequentially the first switch SW1, the first electrode switch ES_{1_1} (the conductive one), the electrode E₁, the living body 2 (which is in contact with the electrode), the electrode E₂, the second electrode switch ES_{2_2} (the conductive one) and the fourth switch SW4, and the current I generated by the voltage source Vs may stimulate the tissue of the living body 2.

For another example, as illustrated in FIG. 2B, the processing device 12 inputs the first input port P₁ with the second level voltage V2, accordingly the first non-inverter 1111 outputs the control signal to the first driver 1112 according to the second level voltage V2, the first driver 1112 controls the first switch SW1 to turn off, the first inverter 1121 outputs the control signal to the second driver 1122 according to the second level voltage V2, and the second driver 1122 controls the second switch SW2 to turn on. The processing device 12 inputs the second input port P₂ with first level voltage V1, accordingly the second non-inverter 1211 outputs the control signal to the third driver 1212 according to the first level voltage V1, the third driver 1212 controls the third switch SW3 to turn on, the second inverter 1221 outputs the control signal to the fourth driver 1222 according to the first level voltage V1, and the fourth driver 1222 controls the fourth switch SW4 to turn off. As a result, the voltage source Vs may form an electrical stimulation path (from high potential to low potential) through sequentially the third switch SW3, at least one conductive one of the second electrode switches ES_{2_1} to ES_{2_N} that is turned on, the corresponding electrode, at least one conductive one of the first electrode switches ES_{1_1} to ES_{1_N} that is turned on and the second switch SW2.

In this embodiment, as illustrated in FIG. 2B, the voltage source Vs forms an electrical stimulation path through sequentially the third switch SW3, the second electrode switch ES_{2_1} (the conductive one), the electrode E₁, the living body 2 (which is in contact with the electrode), the electrode E₂, the first electrode switch ES_{1_2} (the conductive one) and the second switch SW2, the current I generated by the voltage source Vs may stimulate the tissue of the living body 2.

In another embodiment, as long as one of the first switch SW1 and the second switch SW2 may be controlled to be turned on and the other of the first switch SW1 and the second switch SW2 is turned off, the embodiment of the present disclosure is not limited to the circuit designs of the first driving circuit 111 and the second driving circuit 112. Similarly, as long as one of the third switch SW3 and the fourth switch SW4 may be controlled to be turned on and the other of the third switch SW3 and the fourth switch SW4 to be turned off, the embodiment of the present disclosure does not limit the circuit designs of the third driving circuit 121 and the fourth driving circuit 122. In other embodiments, the electrical stimulation device 100 may omit the first driving circuit 111, the second driving circuit 112, the third driving circuit 121 and the fourth driving circuit 122, and the processing device 12 may independently control the first switch SW1 to turn on or off, the second switch SW2 to turn on or off, the third switch SW3 to turn on or off and the fourth switch SW4 to turn on or off.

In addition, the processing device 12 is electrically connected to the first electrode switches ES_{1_1} to ES_{1_N} and the second electrode switches ES_{2_1} to ES_{2_N} to control at least one of the first electrode switches ES_{1_1} to ES_{1_N} to be turned on and at least one of the second electrode switches ES_{2_1} to ES_{2_N} to be turned on for forming the electrical stimulation path. By turning on different first electrode switches and/or turning on different second electrode switches, different electrical stimulation paths and/or a plurality of the combinations of the electrical stimulation paths may be obtained. By turning on different numbers of the first electrode switches and/or turning on different numbers of the second electrode switches, different electrical stimulation paths and/or a plurality of the combinations of the electrical stimulation paths may also be obtained. Further examples are given below.

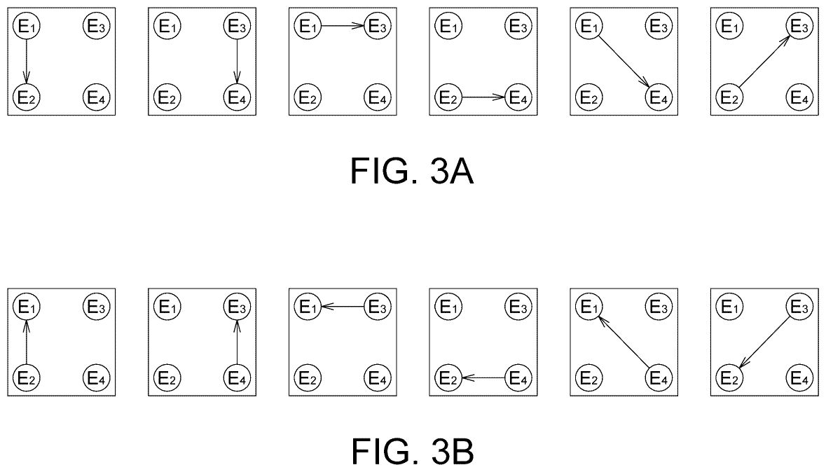

Referring to FIGS. 3A to 3B, FIG. 3A illustrates a schematic diagram of a first electrical stimulation mode according to an embodiment of the present disclosure, and FIG. 3B illustrates a schematic diagram of a second electrical stimulation mode according to an embodiment of the present disclosure.

As illustrated in FIG. 3A, in the first electrical stimulation mode, taking 4 electrodes (N is equal to 4) as an example, the processing device 12 controls one of the first electrode switches ES_{1_1} to ES_{1_N} to be turned on and the others of the first electrode switches ES_{1_1} to ES_{1_N} to be turned off and controls one of the second electrode switches ES_{2_1} to ES_{2_N} to be turned on and the others of the second electrode switches ES_{2_1} to ES_{2_N} to be turned off, and the processing device 12 inputs the first input port P₁ with the first standard voltage V1, and inputs the second input port P₂ with the second standard voltage V2, so that the current may sequentially travel through one of the electrodes E₁ to E_{N} and another of the electrodes E₁ to E_{N} (i.e., one electrode to one electrode) along a direction of the high potential to low potential direction. As a result, six electrical stimulation paths may be obtained.

As illustrated in FIG. 3B, in the second electrical stimulation mode, taking 4 electrodes (N is equal to 4) as an example, the processing device 12 controls one of the first electrode switches ES_{1_1} to ES_{1_N} to be turned on and the others of the first electrode switches ES_{1_1} to ES_{1_N} to be turned off, and controls one of the second electrode switches ES_{2_1} to ES_{2_N} to be turned on and the others of the second electrode switches ES_{2_1} to ES_{2_N} to be turned off, and the processing device 12 inputs the second level voltage V2 to the first input port P₁ and inputs the first level voltage V1 into the second input port P₂, so that the current may sequentially travel through one of the electrodes E₁ to E_{N} and another of the electrodes E₁ to E_{N} along a direction of the high potential to low potential direction (i.e., one electrode to one electrode). As a result, six electrical stimulation paths may be obtained.

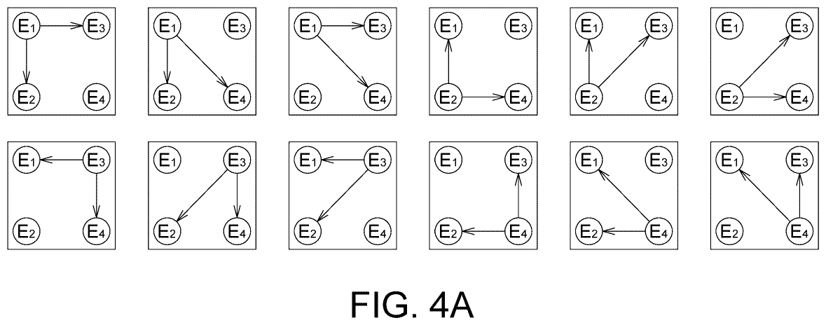

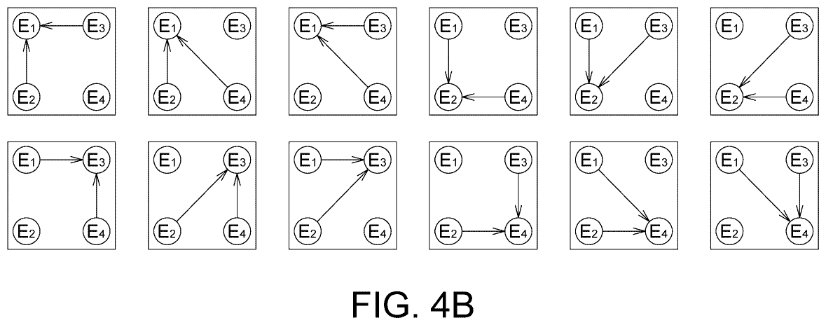

Referring to FIGS. 4A to 4B, FIG. 4A illustrates a schematic diagram of a third electrical stimulation mode according to an embodiment of the present disclosure, and FIG. 4B illustrates a schematic diagram of a fourth electrical stimulation mode according to an embodiment of the present disclosure.

As illustrated in FIG. 4A, in the third electrical stimulation mode, taking 4 electrodes (N is equal to 4) as an example, the processing device 12 controls one of the first electrode switches ES_{1_1} to ES_{1_N} to be turned on and the others of the first electrode switches ES_{1_1} to ES_{1_N} to be turned off, and controls two of the second electrode switches ES_{2_1} to ES_{2_N} to be turned on and the others of the second electrode switches ES_{2_1} to ES_{2_N} to be turned off, and the processing device 12 inputs the first level voltage V1 to the first input port P₁ and inputs the second level voltage V2 into the second input port P₂, so that the current may sequentially travel through one of the electrodes E₁ to E_{N} and another two of the electrodes E₁ to E_{N} along a direction of the high potential to low potential direction (i.e., one electrode to two electrodes). As a result, twelve electrical stimulation paths may be obtained.

As illustrated in FIG. 4B, in the fourth electrical stimulation mode, taking 4 electrodes (N is equal to 4) as an example, the processing device 12 controls two of the second electrode switches ES_{2_1} to ES_{2_N} to be turned on and the others of the second electrode switches ES_{2_1} to ES_{2_N} to be turned off, and controls one of the first electrode switches ES_{1_1} to ES_{1_N} to be turned on and the others of the first electrode switches ES_{1_1} to ES_{1_N} to be turned off, and the processing device 12 inputs the first standard voltage V1 to the second input port P₂ and inputs the second level voltage V2 to the first input port P₁, so that the current may sequentially travel through two of the electrodes E₁ to E_{N} and another of the electrodes E₁ to E_{N} along a direction of the high potential to low potential direction (i.e., one electrode to two electrodes). As a result, twelve electrical stimulation paths may be obtained.

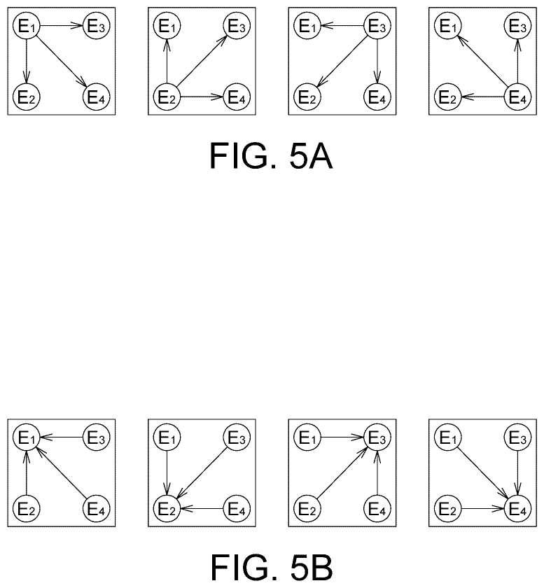

Referring to FIGS. 5A to 5B, FIG. 5A illustrates a schematic diagram of a fifth electrical stimulation mode according to an embodiment of the present disclosure, and FIG. 5B illustrates a schematic diagram of a sixth electrical stimulation mode according to an embodiment of the present disclosure.

As illustrated in FIG. 5A, in the fifth electrical stimulation mode, taking 4 electrodes (N is equal to 4) as an example, the processing device 12 controls one of the first electrode switches ES_{1_1} to ES_{1_N} to be turned on and the others of the first electrode switches ES_{1_1} to ES_{1_N} to be turned off, and controls three of the second electrode switches ES_{2_1} to ES_{2_N} to be turned on and the others of the second electrode switches ES_{2_1} to ES_{2_N} to be turned off, and the processing device 12 inputs the first level voltage V1 to the first input port P₁ and inputs the second level voltage V2 to the second input port P₂, so that the current may sequentially travel through one of the electrodes E₁ to E_{N} and another three of the electrodes E₁ to E_{N} along a direction of the high potential to low potential direction (i.e., one electrode to three electrodes). As a result, four electrical stimulation paths may be obtained.

As illustrated in FIG. 5B, in the sixth electrical stimulation mode, taking 4 electrodes (N is equal to 4) as an example, the processing device 12 controls three of the second electrode switches ES_{2_1} to ES_{2_N} to be turned on and the others of the second electrode switches ES_{2_1} to ES_{2_N} to be turned off, and controls one of the first electrode switches ES_{1_1} to ES_{1_N} to be turned on and the others of the first electrode switches ES_{1_1} to ES_{1_N} are turned off, and the processing device 12 inputs the first standard voltage V1 to the second input port P₂ and inputs the second level voltage V2 to the first input port P₁, so that the current may sequentially travel through three of the electrodes E₁ to E_{N} and another of the electrodes E₁ to E_{N} along a direction of the high potential to low potential direction (i.e., one electrode to three electrodes). As a result, four electrical stimulation paths may be obtained.

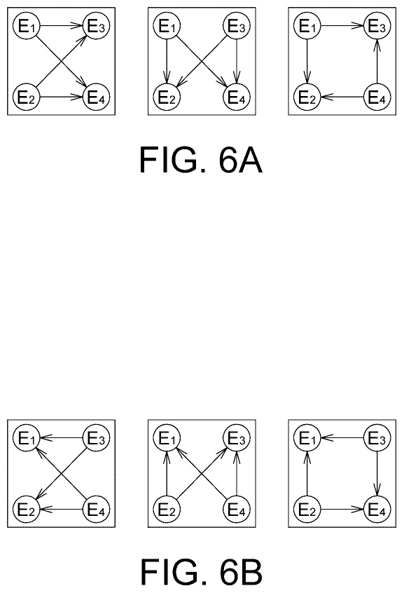

Referring to FIGS. 6A to 6B, FIG. 6A illustrates a schematic diagram of a seventh electrical stimulation mode according to an embodiment of the present disclosure, and FIG. 6B illustrates a schematic diagram of an eighth electrical stimulation mode according to an embodiment of the present disclosure.

As illustrated in FIG. 6A, in the seventh electrical stimulation mode, taking 4 electrodes (N is equal to 4) as an example, the processing device 12 controls two of the first electrode switches ES_{1_1} to ES_{1_N} to be turned on and the others of the first electrode switches ES_{1_1} to ES_{1_N} to be turned off, and controls two of the second electrode switches ES_{2_1} to ES_{2_N} to be turned on and the others of the second electrode switches ES_{2_1} to ES_{2_N} to be turned off, and the processing device 12 inputs the first level voltage V1 to the first input port P₁ and inputs the second level voltage V2 to the second input port P₂, so that the current may sequentially travel through two of the electrodes E₁ to E_{N} and another two of the electrodes E₁ to E_{N} along a direction of the high potential to low potential direction (i.e., two electrodes to two electrodes). As a result, three electrical stimulation paths may be obtained.

As illustrated in FIG. 6B, in the eighth electrical stimulation mode, taking 4 electrodes (N is equal to 4) as an example, the processing device 12 controls two of the second electrode switches ES_{2_1} to ES_{2_N} to be turned on and the others of the second electrode switches ES_{2_1} to ES_{2_N} to be turned off, and controls two of the first electrode switches ES_{1_1} to ES_{1_N} to be turned on and the others of the first electrode switches ES_{1_1} to ES_{1_N} to be turned off, and the processing device 12 inputs the first level voltage V1 to the second input port P₂ and inputs the second level voltage V2 to the first input port P₁, so that the current may sequentially travel through two of the electrodes E₁ to E_{N} and another two of the electrodes E₁ to E_{N} along a direction of the high potential to low potential direction (i.e., two electrodes to two electrodes). As a result, three electrical stimulation paths may be obtained.

The electrical stimulation path of the embodiment of the present disclosure is not limited by FIGS. 3A to 6B. By switching the first electrode switches ES_{1_i} of the first electrode switches ES_{1_1} to ES_{1_N} and switching the second electrode switches ES_{2_j} of the second electrode switches ES_{2_1} to ES_{2_N}, a variety of different electrical stimulation paths may be obtained, wherein the subscripts i and j are positive integers between 1 and N, but i is not equal to j. In other words, the electrical stimulation device 100 of the embodiment of the present disclosure may obtain a variety of different electrical stimulation paths and/or obtain a mesh (or surface-shaped) electrical stimulation path (for example, the paths covers a surface area) by using a small number of electrodes. Furthermore, in case of obtaining the same number of permutations and combinations of electrical stimulation paths, the electrical stimulation device 100 of the present embodiment only requires 4 electrodes, while the conventional electrical stimulation device requires 12 electrodes for obtaining the same number of permutations and combinations. In addition, in an embodiment, the N electrodes E₁ to E_{N} may be disposed corresponding to at least one corner and/or at least one side of a polygon, wherein the polygon is, for example, a triangle, a square, a rectangle, a pentagon or other types of polygons; or N electrodes E₁ to E_{N} may be arranged corresponding to a perimeter of a circle; or the N electrodes E₁ to E_{N} may be arranged corresponding to a perimeter of an ellipse; or the N electrodes E₁ to E_{N} may be arranged in an L×K array shape, wherein L and K are positive integers equal to or greater than 2, and L and K may be equal or different; or the N electrodes E₁ to E_{N} may be arranged along a straight line, a curve or a combination thereof. Different electrode arrangements may obtain different electrical stimulation distribution areas.

In an embodiment, electrical stimulation schemes corresponding to different physiological signals may be different, and multiple electrical stimulation schemes may be pre-stored in the physiological signal detection and electrical stimulation system 10, such as the processing device 12. The processing device 12 may determine the corresponding electrical stimulation plan based on the physiological signal S. An electrical stimulation protocol may include at least one electrical stimulation sequence. In an electrical stimulation sequence, the current lasts for an electrical stimulation time by using a pulse width, an electrical stimulation frequency and an electrical stimulation intensity along an electrical stimulation path, and the current may stimulate along a one-way path or a bidirectional path. In an embodiment, the electrical stimulation time may range between, for example, 1 second and 10 seconds, the pulse width may range between, for example, 10 microseconds and 500 microseconds, and the electrical stimulation frequency may range between, for example, between 10 Hz and 400 Hz., and the electrical stimulation intensity may range between, for example, between 0 and 100 milliamperes, but the foregoing numerical range is not intended to limit the embodiments of the present disclosure. In different two electrical stimulation timing sequences, at least one of the pulse width, the electrical stimulation frequency, the electrical stimulation intensity, the electrical stimulation path, the electrical stimulation time and the one-way electrical stimulation and/or the bidirectional electrical stimulation may be different.

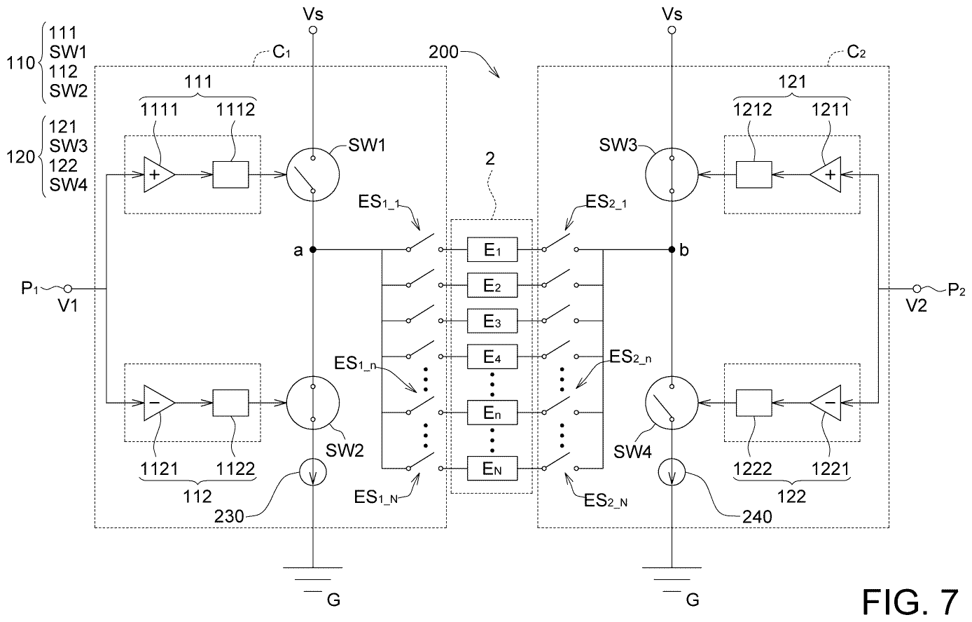

Referring to FIG. 7, FIG. 7 illustrates a circuit schematic diagram of an electrical stimulation device 200 according to another embodiment of the present disclosure.

As illustrated in FIG. 7, the electrical stimulation device 200 includes an electrical stimulation circuit. The electrical stimulation circuit includes N electrodes E₁ to E_{N}, the first circuit assembly C1, the second circuit assembly C2, a first current source 230 and a second current source 240. The first circuit assembly C1 includes N first electrode switches ES_{1_1} to ES_{1_N} and a first control module 110. The second circuit assembly C2 includes N second electrode switches ES_{2_1} to ES_{2_N} and the second control module 120. N is a positive integer equal to or greater than 2. N first electrode switches ES_{1_1} to ES_{1_N} are coupled to N electrodes E₁ to E_{N} respectively. N second electrode switches ES_{2_1} to ES_{2_N} are coupled to N electrodes E₁ to E_{N} respectively. The processing device 12 is configured to determine the action modes of the N first electrode switches ES_{1_1} to ES_{1_N}, the N second electrode switches ES_{2_1} to ES_{2_N}, the first control module 110 and the control module 120 according to the physiological signal S (the physiological signal S is illustrated in FIG. 1B). The N first electrode switches ES_{1_1} to ES_{1_N} share the contact point a with the first control module 110, and the N second electrode switches ES_{2_1} to ES_{2_N} share the contact point b with the second control module 120. As a result, through the action modes of the N first electrode switches ES_{1_1} to ES_{1_N}, the N second electrode switches ES_{2_1} to ES_{2_N}, the first control module 110 and the second control module 120, the current path may be controlled to obtain the expected electrical stimulation effects.

Different from the electrical stimulation device 100, the electrical stimulation device 200 further includes a first current source 230 and a second current source 240. The first current source 230 is coupled to the ground potential G and the second switch SW2, and the second current source 240 is coupled to the ground potential G and the fourth switch SW4. The current source may provide the stable current output for providing the better electrical stimulation effect (current stimulation effect on the living body is more significantly than voltage stimulation effect).

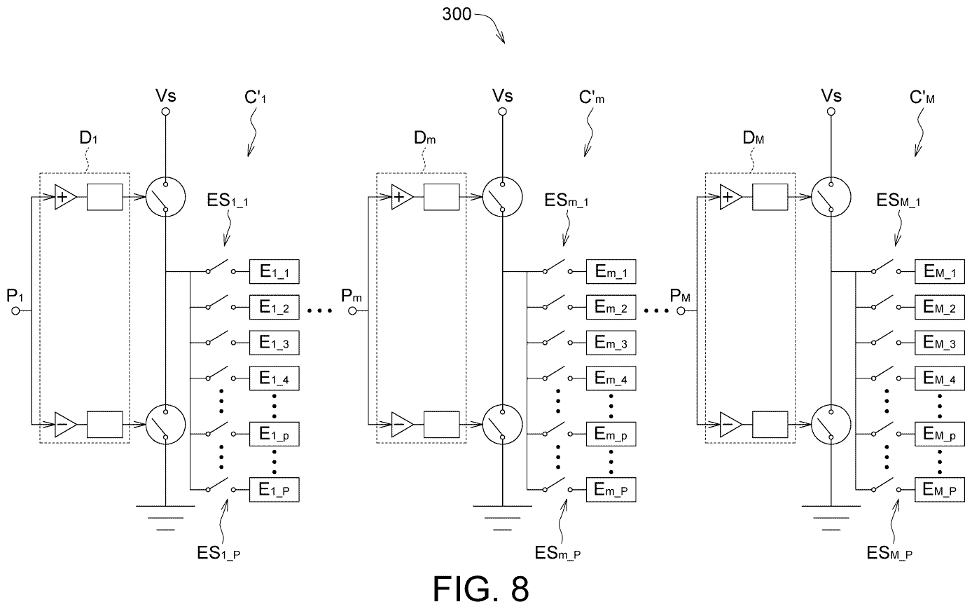

Referring to FIG. 8, FIG. 8 illustrates a circuit schematic diagram of an electrical stimulation device 300 according to another embodiment of the present disclosure.

As illustrated in FIG. 8, the electrical stimulation device 300 includes an electrical stimulation circuit. The electrical stimulation circuit includes M circuit assemblies C′₁ to C′_{M}, wherein M is, for example, a positive integer equal to or greater than 2. The circuit assembly C′ₘ includes P electrode switches ES_{m_1} to ES_{m_P}, a control module Dm and P electrodes E_{m_1} to E_{m_P}, wherein m is a positive integer between 1 and M, P is a positive integer greater than or equal to 2, and the value of P may be less than, equal to or greater than the aforementioned value of N. In case of m being equal to 1, the circuit assembly C′₁ includes P electrode switches ES_{1_1} to ES1_P, the control module D₁ and P electrodes E_{1_1} to E_{1_P}. In case of m being equal to M, the circuit assembly C′_{M} includes P electrode switches ES_{M_1} to ES_{M_P}, the control module DM and P electrodes E_{M_1} to E_{M_P}. In addition, each control module Dm includes the structures the same as or similar to that of the aforementioned first control module 110 or second control module 120, and they will not be repeated again here.

In an embodiment, current may be transmitted from one of the circuit assemblies C′₁ to C′ₘ to at least one of the others of the circuit assemblies C′₁ to C′ₘ through the living body 10 (not illustrated in FIG. 8). For example, current may be transmitted from at least one of the P electrodes E_{m_1} to E_{m_P} of one of the circuit assemblies C′₁ to C′ₘ to at least one of the P electrodes E_{m_1} to E_{m_P} of at least one of the others of the circuit assemblies C′₁ to C′_{M} through the living body 10.

In an embodiment, two connected circuit assemblies may share a common electrode. For example, at least one of the P electrodes E_{m_1} to E_{m_P} of one of the circuit assemblies C′₁ to C′_{M} and at least one of the P electrodes E_{m_1} to E_{m_P} of at least one of the others of the circuit assemblies C′₁ to C′_{M} are the same electrode (i.e., a common electrode), and accordingly it may reduce the number of electrodes. In addition, a plurality of the current paths may travel from one electrode to a plurality of the electrodes (from the high potential to the low potential), or a plurality of the current paths may travel from a plurality of the electrodes to a plurality of the electrodes (from the high potential to the low potential), or a plurality of the current paths may travel from a plurality of the electrodes to one electrode (from the high potential to the low potential) to form a mesh current path of arbitrary combination.

In summary, a physiological signal detection and electrical stimulation system is provided according to an embodiment of the present disclosure, its electrical stimulation device and method and physiological signal detection and electrical stimulation system may detect the physiological signals of the living body and apply the corresponding electrical stimulation respond to physiological signals based on the physiological signals. The detection of the physiological signals and the application of the electrical stimulation are integrated into the same physiological signal detection and electrical stimulation system, which is a complete solution that may meet the needs of clinical therapists and patients for home exercise training. In addition, the electrical stimulation device includes two circuit assemblies, and the two circuit assemblies share at least one electrode. As a result, the electrical stimulation device may obtain a variety of different electrical stimulation paths and/or obtain a mesh (or surface) electrical stimulation path (for example, the path covers an area) uses a few electrodes. In an embodiment, the circuit assembly is, for example, an H half-bridge circuit.

What is claimed is:

1. A physiological signal detection and electrical stimulation system, comprising:
a physiological signal processing unit configured to generate a physiological signal;
an electrical stimulation device, comprising:
N electrodes, wherein N is a positive integer equal to or greater than 2;
a first circuit assembly, comprising:
N first electrode switches coupled to the N electrodes respectively; and
a first control module coupled to the N first electrode switches; and
a second circuit assembly, comprising:
N second electrode switches coupled to the N electrodes respectively; and
a second control module coupled to the N second electrode switches;
a processing device electrically connected to the electrical stimulation device and the physiological signal processing unit, and configured to:
control the N first electrode switches, the N second electrode switches, the first control module and the second control module to generate a current flowing along an electrical stimulation path according to the physiological signal;
wherein the N first electrode switches and the first control module share a contact point, and the N second electrode switches and the second control module share another contact point.

2. The physiological signal detection and electrical stimulation system according to claim 1, wherein the first control module comprises:
a first switch;
a second switch;
a first driving circuit electrically connected to the first switch to control the first switch to be turned on or off; and
a second driving circuit electrically connected to the second switch to control the second switch to be turned on or off;
wherein the second control module comprises:
a third switch;
a fourth switch;
a third driving circuit electrically connected to the third switch to control the third switch to be turned on or off; and
a fourth driving circuit electrically connected to the fourth switch to control the fourth switch to be turned on or off.

3. The physiological signal detection and electrical stimulation system according to claim 2, wherein the processing device is further configured to:
control one of the first switch and the second switch to be turned on and the other of the first switch and the second switch to be turned off according to one of a first level voltage and a second level voltage; and
control one of the third switch and the fourth switch to be turned on and the other of the third switch and the fourth switch to be turned off according to another of the first level voltage and the second level voltage.

4. The physiological signal detection and electrical stimulation system according to claim 2, wherein the first switch and the third switch are electrically connected to a voltage source, and the second switch and the fourth switch are electrically connected to a ground potential.

5. The physiological signal detection and electrical stimulation system according to claim 2, wherein the electrical stimulation device further comprises:
a first current source coupling the second switch with a ground potential; and
a second current source coupling the fourth switch with the ground potential.

6. The physiological signal detection and electrical stimulation system according to claim 1, wherein the processing device is further configured to:
control at least one of the N first electrode switches to be turned on and the others of the N first electrode switches to be turned off; and
control at least one of the N second electrode switches to be turned on and the others of the N second electrode switches to be turned off.

7. An electrical stimulation device, comprising:
N electrodes, wherein N is a positive integer equal to or greater than 2;
a first circuit assembly, comprising:
N first electrode switches coupled to the N electrodes respectively; and
a first control module coupled to the N first electrode switches; and
a second circuit assembly, comprising:
N second electrode switches coupled to the N electrodes respectively; and
a second control module coupled to the N second electrode switches;
wherein the N first electrode switches and the first control module share a contact point, and the N second electrode switches and the second control module share another contact point.

8. The electrical stimulation device according to claim 7, wherein the first control module comprises:
a first switch;
a second switch;
a first driving circuit electrically connected to the first switch to control the first switch to be turned on or off; and
a second driving circuit electrically connected to the second switch to control the second switch to be turned on or off;
wherein the second control module comprises:
a third switch;
a fourth switch;
a third driving circuit electrically connected to the third switch to control the third switch to be turned on or off; and
a fourth driving circuit electrically connected to the fourth switch to control the fourth switch to be turned on or off.

9. The electrical stimulation device according to claim 8, wherein the first switch and the third switch are electrically connected to a voltage source, and the second switch and the fourth switch are electrically connected to a ground potential.

10. The electrical stimulation device according to claim 8, further comprising:
a first current source coupling the second switch with a ground potential; and
a second current source coupling the fourth switch with the ground potential.

11. A physiological signal detection and electrical stimulation method, comprising:
generating a physiological signal by a physiological signal processing unit of a physiological signal detection and electrical stimulation system, wherein the physiological signal detection and electrical stimulation system further comprises an electrical stimulation device and a processing device, the electrical stimulation device comprises N electrodes, a first circuit assembly and a second circuit assembly, the first circuit assembly comprises N first electrode switches and a first control module, the second circuit assembly comprises N second electrode switches and a second control module, wherein N is a positive integer equal to or greater than 2, the N first electrode switches are coupled to the N electrodes respectively, the N second electrode switches are coupled to the N electrodes respectively, the first control module is coupled to the N first electrode switches, the second control module is coupled to the N second electrode switches, the N first electrode switches and the first control module share a contact point, the N second electrode switches and the second control module share another contact point, and the processing device is electrically connected to the electrical stimulation device and the physiological signal processing unit; and
controlling the N first electrode switches, the N second electrode switches, the first control module and the second control module to generate a current flowing along an electrical stimulation path according to the physiological signal.

12. The physiological signal detection and electrical stimulation method according to claim 11, wherein the first control module comprises a first switch, a second switch, and a first driving circuit electrically connected to the first switch and a second driving circuit electrically connected to the second switch, the second control module comprises a third switch, a fourth switch, and a third driving circuit electrically connected to the third switch and a fourth driving circuit electrically connected to the fourth switch; the electrical stimulation method further comprises:
controlling the first switch to be turned on or off by the first driving circuit;
controlling the second switch to be turned on or off by the second driving circuit;
controlling the third switch to be turned on or off by the third driving circuit; and
controlling the fourth switch to be turned on or off by the fourth driving circuit.

13. The physiological signal detection and electrical stimulation method according to claim 12, further comprising:
controlling one of the first switch and the second switch to be turned on and the other of the first switch and the second switch to be turned off according to one of a first level voltage and a second level voltage by the processing device; and
controlling one of the third switch and the fourth switch to be turned on and the other of the third switch and the fourth switch to be turned off according to another of the first level voltage and the second level voltage by the processing device.

14. The physiological signal detection and electrical stimulation method according to claim 12, wherein the first switch and the third switch are electrically connected to a voltage source, and the second switch and the fourth switch are electrically connected to a ground potential.

15. The physiological signal detection and electrical stimulation method according to claim 12, wherein the electrical stimulation device further comprises a first current source and a second current source, the first current source couples the second switch with a ground potential, and the second current source couples the fourth switch with the ground potential.

16. The physiological signal detection and electrical stimulation method according to claim 11, further comprising:
controlling at least one of the N first electrode switches to be turned on and the others of the N first electrode switches to be turned off by the processing device; and
controlling at least one of the N second electrode switches to be turned on and the others of the N second electrode switches to be turned off by the processing device.

17. An electrical stimulation device, comprising:
M circuit assemblies, wherein M is a positive integer equal to or greater than 2, and each of the M circuit assemblies comprises:
P electrodes, wherein P is a positive integer equal to or greater than 2;
P electrode switches coupled to the P electrodes respectively; and
a control module coupled to the P electrode switches;
wherein a current is allowed to be transmitted from at least one of the P electrodes of one of the M circuit assemblies to at least one of the P electrodes of at least one of the others of the M circuit assemblies.

18. The electrical stimulation device according to claim 17, wherein at least one of the P electrodes of one of the M circuit assemblies and at least one of the P electrodes of at least one of the M circuit assemblies share the same electrode.
